(19) United States
(12) Patent Application Publication
(10) Pub. No.: US 2017/0173286 A1
(43) Pub. Date: Jun. 22, 2017

(54) TRACHEOSTOMY TUBE ASSEMBLIES, INNER CANNULAE AND METHODS OF MAKING INNER CANNULAE

(71) Applicant: SMITHS MEDICAL INTERNATIONAL LIMITED, Kent (GB)

(72) Inventors: Timothy Bateman, Kent (GB); Stephen James Field, Kent (GB); Mark Andrew Graham, Kent (GB); Andrew Thomas Jeffrey, Kent (GB)

(73) Assignee: SMITHS MEDICAL INTERNATIONAL LIMITED, Kent (GB)

(21) Appl. No.: 15/116,071

(22) PCT Filed: Jan. 14, 2015
(86) PCT No.: PCT/GB2015/000008

§ 371 Date: Aug. 2, 2016

(30) Foreign Application Priority Data
Feb. 5, 2014 (GB) 1401929.3

(51) Int. Cl. A61M 16/04; B29C 45/14

(52) U.S. Cl. CPC A61M 16/0427; A61M 16/0465; A61M 16/0434; B29C 45/14622; A61M 2207/00; B29K 2101/12

(57) ABSTRACT

An inner cannula (20) for a tracheostomy tube assembly includes a thin, inner sheath (22) supported externally by a structural frame (23) having a longitudinal portion (25) and a plurality of ribs (26) extending circumferentially and spaced along the cannula. The inner cannula (20) is inserted within and is removable from an outer tracheostomy tube (1). The inner cannula (20) is made by coating a plastic layer on a core to form the sheath and then placing the sheath on a curved core. The structural frame (23) is then moulded about the sheath (22) in a mould 80.

This invention relates to inner cannulae of the kind for a tracheostomy tube assembly including a structural frame and a sheath extending over the frame.

Tracheostomy tube assemblies commonly include an outer tube and an inner tube or cannula that is a removable fit within the outer tube. The inner cannula can be removed and replaced periodically to ensure that the passage through the assembly does not become blocked by secretions. This avoids the need to remove the outer tube frequently.

The inner cannula presents various problems because it must be thin walled and a close fit within the outer tube so as to provide a large bore and thereby limit the resistance to flow of gas along the assembly. It must, however, also be sufficiently stiff to be inserted in the outer tube without buckling or kinking. WO94/01156 and WO2004/101048 describe inner cannulae made of PTFE. EP1938857 describes an arrangement of tracheostomy tubes and inner cannulae where the hubs of the inner cannulae of different sizes are shaped differently so that they will only fit in the appropriate tracheostomy tube. EP2224985 describes an arrangement for attaching a hub to the shaft of an inner cannula. GB2056285 describes an inner cannula having a wall corrugated both externally and internally and a longitudinal groove or other reinforcement member traversing at least some of the corrugations. U.S. Pat. No. 4,817,598 describes a smooth-walled inner cannula having a ring-pull formation at its rear, machine end. U.S. Pat. No. 5,119,811 describes an inner cannula with a flared patient end and formed of two layers of different materials. U.S. Pat. No. 5,386,826 describes an inner cannula with an outer helical filament or layer of low friction material. U.S. Pat. No. 5,983,895 describes an inner cannula with straight sections at opposite ends joined by an intermediate curved section. U.S. Pat. No. 6,019,753 describes an inner cannula with two elongate regions of different flexibility so that the cannula has a plane of preferential bending. U.S. Pat. No. 6,0197,53 describes an inner cannula having a shaft formed with slots to make it more flexible, the slots being covered by an outer thin sheath. U.S. Pat. No. 6,135,110 describes a curved inner cannula that is retained with the outer tube by means of a rotatable spring fitting.

It is an object of the present invention to provide an alternative inner cannula and tracheostomy tube assembly.

According to one aspect of the present invention there is provided an inner cannula of the above-specified kind, characterised in that the frame provides an outer surface of the cannula and the sheath provides an inner surface of the cannula.

The structural frame is preferably moulded of a stiff plastics material and preferably includes a longitudinal portion and a plurality of rib members extending circumferentially around a part at least of the cannula. The sheath may have a wall thickness between 0.1 mm and 1 mm.

According to another aspect of the present invention there is provided a method of making an inner cannula for a tracheostomy tube assembly including the steps of coating a layer of a first liquid plastics material on the outside of a cylindrical core, allowing the plastics material to cure to form a cylindrical sheath, filling a space in a mould around the outside of the sheath with a second liquid plastics material to bond with the sheath, the space defining with the outside of the sheath the shape of a structural frame, allowing the second plastics material to cure and form the structural frame, and subsequently removing the sheath and bonded structural frame together from the mould.

The cured sheath may be removed from the core and placed on a second core prior to moulding the structural frame about the sheath. The second core may be curved along its length.

According to a further aspect of the present invention there is provided an inner cannula made by a method according to the above other aspect of the present invention.

According to a fourth aspect of the present invention there is provided a tracheostomy tube assembly including an outer tracheostomy tube and an inner cannula according to the above one or further aspect of the present invention, the inner cannula being inserted within the outer tube and being removable therefrom.

A tracheostomy tube assembly including an inner cannula and a method of making an inner cannula, all in accordance with the present invention, will now be described, by way of example, with reference to the accompanying drawings, which are not to scale, and in which:

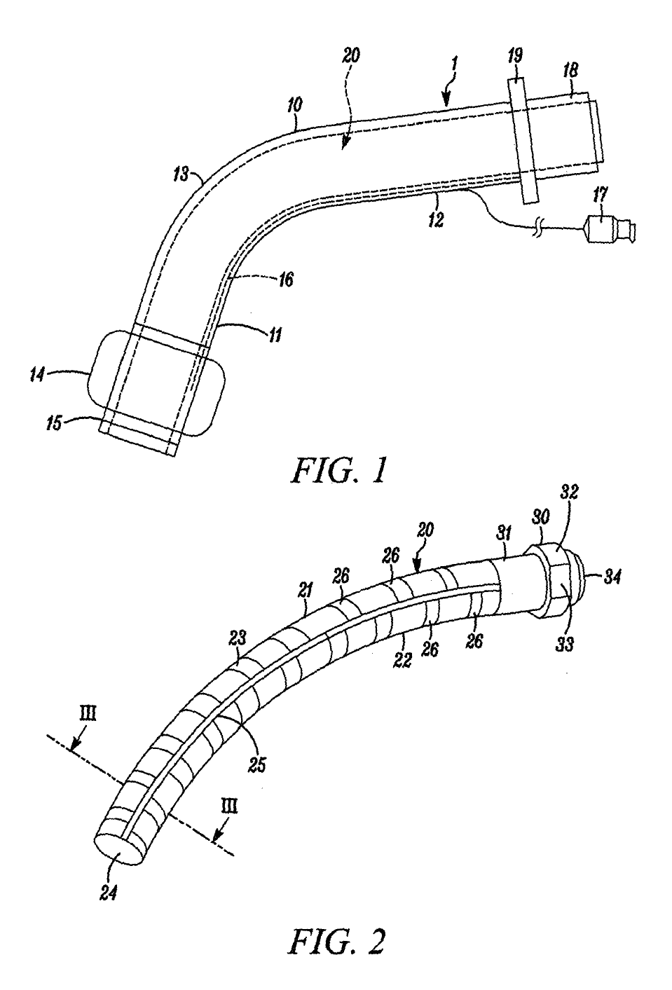

FIG. 1 is a side elevation view of a tracheostomy tube assembly including an inner cannula;

FIG. 2 is a perspective view of the inner cannula;

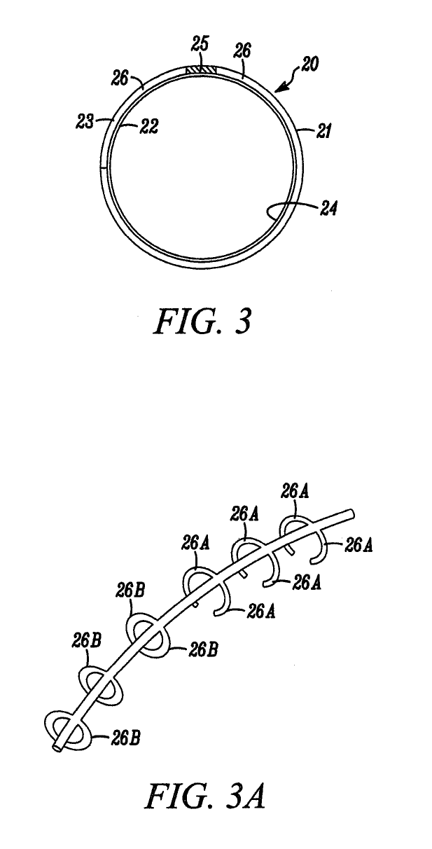

FIG. 3 is a cross-sectional view through the inner cannula along the line of FIG. 2;

FIG. 3A is a perspective view of an alternative form of frame having different arrangements of ribs along its length; and

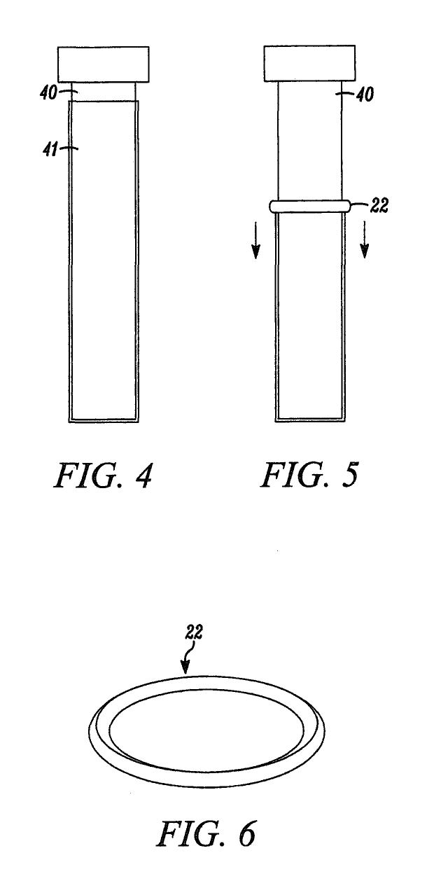

FIGS. 4 to 8 illustrate different stages in the manufacture of the inner cannula.

With reference first to FIG. 1, the tracheostomy tube assembly comprises an outer tracheostomy tube 1 and a removable inner cannula 20 inserted within the outer tube. The outer tube 1 has a shaft 10 with a straight forward section 11, a straight rear section 12 and a curved intermediate section 13 linking the forward and rear sections. An inflatable sealing cuff 14 embraces the forward section 11 close to the patient end 15 of the tube, the cuff being inflated via an inflation lumen 16 and a combined connector and inflation indicator 17. At its rear end, the outer tube 1 has a hub 18 and flange 19 to which a retaining tape can be fastened for securing the tube with the patient's neck. The inside of the hub 18 is formed with keying flats (not shown), of the kind described in EP1938857, adapted to prevent full insertion of an inner cannula of the wrong size. The outer tube 1 could have an internal diameter between about 2 mm and 10 mm, and its length could be between 60 mm and 200 mm.

With reference now also to FIGS. 2 and 3, the inner cannula 20 is formed by a shaft 21 of circular section attached at its rear or machine end with a hub 30. The shaft 21 includes two components, that is, a thin, inner sheath 22 and an outer structural frame 23. The inner sheath 22 provides the inner surface or bore 24 of the cannula 20 and is made of a thin, flexible plastics material with a wall thickness between about 0.1 mm and 1 mm. The sheath 22 may be of any suitable plastics material, such as PVC, polyurethane, polyethylene, polypropylene or other flexible or semi-rigid plastics material. The outer frame 23 is moulded of a stiff plastics material and comprises a spine or longitudinal portion 25 extending along the length of the frame with about ten ribs 26 on each side integrally moulded with the spine. The ribs 26 extend laterally, circumferentially around the frame 23 by about 270° from the spine 25 in a staggered fashion from opposite sides of the spine so that the ribs are interdigitated along the side of the frame opposite the spine. Other arrangements of ribs could be used. The ribs could be aligned and could extend around the entire circumference of the frame as illustrated in FIG. 3A, which shows an arrangement of incomplete, aligned ribs 26A at one end of the frame and at the opposite end of the frame a series of ribs 26B that extend around the entire circumference. The inner surface of the frame 23 is bonded with the outer surface of the sheath 22. The hub 30 has a forward, patient end portion 31 shaped to fit into a 15 mm connector. To the rear of the forward portion 31 the hub 30 has a keying portion 32 provided with flats 33 of the kind described in EP1938857 adapted to fit with corresponding formations in the hub 18 of the outer tube 1. At its rear end, the inner cannula 20 has a ring-pull formation 34 of the kind described in U.S. Pat. No. 4,817,598, which facilitates removal of the inner cannula from the outer tube 1 after use.

The outer diameter of the shaft 21 across the frame 23 is matched with the internal diameter of the outer tube 1 so that the inner cannula 20 can be freely slid into and removed from the outer tube. Contact of the inner cannula 20 with the inside of the tube is limited to the outer surface of the frame 23, which has a relatively small surface area compared with conventional inner cannulae. This low area of contact keeps friction between the cannula 20 and the outer tube 1 to a minimum, thereby facilitating its insertion and removal. The sheath 22 on the inner surface of the cannula 20 gives the cannula a smooth bore 24, which reduces turbulence of gas flow along the assembly and also enables devices (such as a suction catheter or visualisation device) to be slid along the bore of the cannula without snagging. By manufacturing the shaft 21 of the inner cannula 20 from the combination of an inner sheath 22 and outer frame 23 it is possible to achieve the necessary strength and axial rigidity for the cannula and also minimise the wall thickness of the cannula, thereby ensuring a minimal obstruction to flow of gas along the assembly.

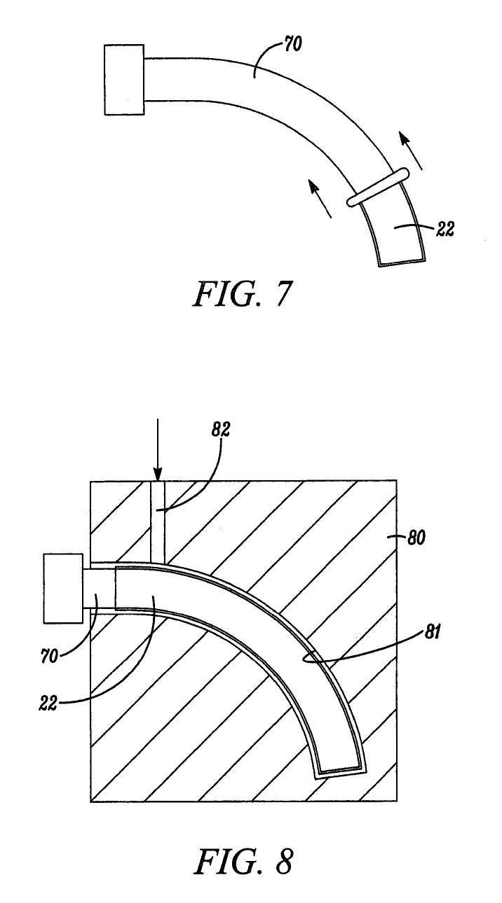

The inner cannula 20 is preferably made in the manner shown in FIGS. 4 to 7. The sheath 21 is made in the manner shown in FIGS. 4 to 6 using a cylindrical core 40 that is coated with a thin layer 41 of plastics material such as by dipping, spraying, moulding or any other conventional technique. The layer 41 is allowed to cure, which may involve heat treatment, to form a closed-end sheath 22. The sheath 22 is then rolled off by rolling from the open end down to the closed end and the rolled up sheath is removed from the core 40 in the rolled up form shown in FIG. 6. The rolled-up sheath 22 is then loaded on a second core 70 shown in FIG. 7, which is of circular section and is curved to the desired shape of the inner cannula 20. The sheath 22 is rolled onto the second core 70 by unrolling from one end of the core. The second core 70 with the loaded sheath 22 is then placed within an outer mould tool 80 as shown in FIG. 8. The inner surface 81 of the mould tool 80 follows closely the outer surface of the core 70 apart from recesses defining the shape of the outer structural frame 23. A plastics material for forming the frame 23 is injected in liquid form into the spaces between the outside of the sheath 22 and the inside 81 of the outer mould tool 80 via a feed passage 82 to fill the recesses and bond with the outside surface of the sheath and form the shaft 21. After the frame material has cured, the mould inner core 70 with the shaft 21 loaded on it is removed from the outer mould tool 80 and the shaft 21 is then removed from the core. The closed end of the sheath 22 is removed and the hub 30 is attached to the rear end of the shaft 21 such as by means of an adhesive or bonding substance, or the hub could be overmoulded onto the shaft. Alternatively, the hub could be moulded with the frame. Instead of removing the sheath 22 from the first core 40 it could be left on this core and placed directly in the moulding cavity. In such an arrangement the core would be curved to the desired final shape of the inner cannula.

It will be appreciated that the inner cannula could be made in other ways. For example, it could be made by a two-shot injection moulding process where the inner sheath is moulded first and then overmoulded with the frame in a second cavity. The small thickness of the inner sheath makes it the more difficult to mould.

1-9. (canceled)

10. An inner cannula for a tracheostomy tube assembly including a structural frame and a sheath extending over the frame, characterized in that the sheath provides an inner surface of the cannula, and that the outer surface of the cannula is provided by the frame such that a relatively small surface area on the outside of the cannula is in contact with the inside of the tracheostomy tube.

11. An inner cannula according to claim 10, characterized in that the structural frame is moulded of a stiff plastics material.

12. An inner cannula according to claim 10, characterized in that the structural frame includes a longitudinal portion and a plurality of rib members extending circumferentially around a part at least of the cannula.

13. An inner cannula according to claim 10, characterized in that the sheath has a wall thickness between 0.1 mm and 1 mm.

14. A method of making an inner cannula for a tracheostomy tube assembly including the steps of coating a layer of a first liquid plastics material on the outside of a cylindrical core, allowing the plastics material to cure to form a cylindrical sheath, filling a space in a mould around the outside of the sheath with a second liquid plastics material to bond with the sheath, the space defining with the outside of the sheath the shape of a structural frame, allowing the second plastics material to cure and form the structural frame, and subsequently removing the sheath and bonded structural frame together from the mould.

15. A method according to claim 14, characterized in that the cured sheath is removed from the core and placed on a second core prior to moulding the structural frame about the sheath.

16. A method according to claim 15, characterized in that the second core is curved along its length.

17. An inner cannula for a tracheostomy tube assembly made by coating a layer of a first liquid plastics material on the outside of a cylindrical core, allowing the plastics material to cure to form a cylindrical sheath, filling a space in a mould around the outside of the sheath with a second liquid plastics material to bond with the sheath, the space defining with the outside of the sheath the shape of a structural frame, allowing the second plastics material to cure and form the structural frame, and subsequently removing the sheath and bonded structural frame together from the mould.

18. A tracheostomy tube assembly including an outer tracheostomy tube and an inner cannula having a structural frame and a sheath extending over the frame, the sheath providing an inner surface of the cannula, an outer surface of the cannula provided by the frame such that a relatively small surface area on the outside of the cannula is in contact with the outer tracheostomy tube, the inner cannula being inserted within the outer tube and being removable therefrom.
